(19) United States
(12) Patent Application Publication
(10) Pub. No.: US 2018/0000727 A1
(43) Pub. Date: Jan. 4, 2018

(54) COMPOSITION AND METHODS TO IMPROVE STABILITY, DOSING, PHARMACODYNAMICS AND PRODUCT SHELF LIFE OF ENDOCANNABINOIDS, PHYTOCANNABINOIDS AND SYNTHETIC CANNABINOIDS DELIVERED BY NASAL INHAER

(71) Applicant: Michael Willinsky, Hamilton, Ontario (CA)

(72) Inventor: Michael Willinsky, Hamilton, Ontario (CA)

(21) Appl. No.: 15/545,500

(22) PCT Filed: Jan. 18, 2016
(86) PCT No.: PCT/CA2016/000010

§ 371 Date: Jul. 21, 2017

Related U.S. Application Data
(60) Provisional application No. 62/125,392, filed on Jan. 21, 2015.

(51) Int. Cl. A61K 9/00; A61K 47/12; A61K 31/352; A61K 9/14; A61K 9/12; A61K 47/44; A61K 36/18

(52) U.S. Cl. CPC A61K 9/008; A61K 47/44; A61K 47/12; A61K 36/18; A61K 9/0043; A61K 9/14; A61K 9/124; A61K 31/352

(57) ABSTRACT

An inhaler-delivery-device-packaged homogenate of solid heterogeneous-lipid particulates carrying lipophilic cannabinoid receptor agonists and/or antagonists, wherein the solid heterogeneous-lipid particles comprises: one (or more) lipid(s) whose melting point(s) is (are) substantially above room temperature; in combination with, one (or more) lipid(s) whose melting point(s) is (are) substantially less than room temperature.

This application claims the benefit of U.S. provisional patent No. 62/125,392, filed on Jan. 21, 2015. The entire disclosure of which is incorporated by reference herein.

### FIELD OF THE INVENTION

Aspects of the present invention include and variously relate, (both individually and through combinations thereof), to:

cannabinoid-receptor-centric therapeutics and therapies;

homogenates including solid lipid particulates, methods and apparatus for packaging, distribution and administration of pharmaceutically active agents, but of cannabinoids in particular, as aids against over or under dosing and misuse, and to help overcome certain prior art prejudices against nasal/pharyngeal/esophageal administration routes, and particularly those which appertain to cannabinoids; and,

combinations and methods involving cannabinoids and one or more of opiates such as morphine (a hydrophilic opiate) or methadone (a lipophilic opiate).

### BACKGROUND OF THE INVENTION

Cannabis plants have an extensive history of medicinal usages dating back thousands of years and across many civilizations, (see for example: Ben Amar M (2006). “Cannabinoids in medicine: a review of their therapeutic potential” Journal of Ethnopharmacology (Review) 105 (1-2): 1-25.) “Medical cannabis”, or “medical marijuana”, are express references to the utilization of cannabis and/or its phytocannabinoid constituency to treat disease or improve symptoms. There is an accumulating body of evidence in favor of such usages (along with endocannabinoids or even synthetic cannabinoids), as for example in reducing nausea and vomiting collaterally associated with chemotherapy treatments; improving appetite in people with HIV/AIDS, and treating chronic pain and/or muscle spasms. (see for example: Borgelt, L M; Franson, K L; Nussbaum, A M; Wang, G S (February 2013). “The pharmacologic and clinical effects of medical cannabis.”, Pharmacotherapy 33 (2): 195-209; or, Whiting, P F; Wolff, R F; Deshpande, S; Di Nisio, M; Duffy, S, Hernandez, A V; Keurentjes, J C; Lang, S; Misso, K; Ryder, S; Schmidlkofer, S; Westwood, M; Kleijnen, J (23 Jun. 2015). “Cannabinoids for Medical Use: A Systematic Review and Meta-analysis.” JAMA 313 (24): 2456-2473.)

Research performed over the last 30 years has provided scientific and medical evidence that cannabinoid preparations are useful in treating a variety of conditions including pain, glaucoma and nausea, Physicians Desk Reference. 59th Ed. (2005). While the medical efficacy of cannabinoids is now generally accepted, many Physicians remain confused about the many strains and constituents of Cannabis. To add to the confusion, cannabinoids can be smoked, vaporized, taken orally, sublingually, buccally or rectally with unacceptably wide variation in the rate of absorption, onset and duration of action. This contributes to the feeling shared by most Physicians that they are ill equipped to prescribe cannabinoids and/or marijuana for medical purposes.

U.S. Patent No. 2003/0100602 proposes oral administration of dronabinol, a synthetic cannabinoid, to stimulate appetite and reduce weight loss in HIV patients. Administration by suppository, transdermal, sublingual, pulmonary intranasal and injection are also mentioned. While administration of cannabinoids orally can be useful, there remain challenges. Orally administered cannabinoids are absorbed by blood which perfuses the hepatic portal system of the liver where first pass hepatic uptake of cannabinoids results in rapid metabolism. in the case of Δ9-THC, only 10% of an oral dose reaches the circulation unchanged. Oral delivery of cannabinoids presents other challenges as the same THC dose yields different plasma levels between patients. Patients can absorb less of the drug or metabolize more, with resultant diminished or no therapeutic benefit. Further, clinical effects are not experienced for hours after oral administration.

U.S. Patent No. 2003/0021752 attempts to address this problem with a mucosal delivery system for lipophilic cannabinoids using an emulsion which adheres to mucosa causing cannabinoid absorption. However, such traditional delivery systems employed for hydrophilic drugs are inefficient when applied to lipophilic drugs like cannabinoids and result in erratic bioavailability.

U.S. Pat. No. 4,464,378 proposes a nasal dosage form of Δ9-THC by suspending the drug in an aqueous system.

U.S. Pat. No. 6,380,175 proposes enhanced delivery of Δ9-THC by nasal dosage of a water soluble pro-drug.

U.S. Patent No. 2003/00033113 proposes administration of cannabinoids as part of an addiction therapy. Transdermal, sublingual and nasal routes of administration are mentioned.

U.S. Patent No. 2004/0186166 proposes cannabinoids for disorders involving peroxizome proleferator-activated receptor gamma and mentions nasal administration.

Pylak et al. (1999) Soc. NeuroSci. Abstr. 25(1):924 reports Δ9-THC administered intranasally at 1.0 to 1.3 mg/kg in a rats produced an analgesic response 15 minutes post administration which lasted 120 minutes. Analgesic effects are compared to ethanol and the endogenous cannabinoid anandamide.

U.S. Pat. No. 4,454,378 describes nasal administration of cannabinoids as a simple spray, ointment, gel or suspension. No examples of THC formulation were described. A person skilled in the art would not expect such a simple THC formulation to be successful on account of the very poor water solubility of cannabinoids.

U.S. Pat. No. 6,383,513 describes nasal administration of a biphasic albumin based microsphere system for cannabinoids to improve absorption for treatment of pain, nausea and appetite stimulation.

U.S. Pat. No. 6,630,507 describes Cannabinoids that have antioxidant and neuroprotective properties not mediated via antagonism of NMDA receptors. This property makes cannabinoids potentially useful in treatment of age related ischemia, ischemic stroke and inflammatory disease.

European Pat. No. EP 1,361,864 describes liquid spray formulations of cannabinoids for use oral administration of medicaments via absorption through sublingual or the buccal mucosa to avoid first pass hepatic uptake.

U.S. Patent No. 2006/0,257,463 describes methods and products for transmucosal oral delivery of cannabinoids wherein said transmucosal preparation is made by incorporating cannabinoids with hot-melt extrusion technology in order to avoid first pass hepatic uptake.

Notwithstanding this history and the trending of more recent scientific insights in support of the benefits of cannabinoid receptor therapies, controversy continues to persist around the medical prescription of canabanoids. Even the American Medical Association, the Minnesota Medical Association, the American Society of Addiction Medicine, among other medical organizations, have issued statements opposing its use, at present, for medicinal purposes.

Not the least of the problems associated with acceptance of cannabinoid receptor therapies has been summarized by Grotenhermen, F., Journal of Cannabis Therapeutics, Vol. 3(1) 2003: Among the reasons for the decline of the medical use of cannabis in the first half of the 20th century were the pharmacokinetic properties of THC in oral preparations (tinctures, fatty extracts). With oral use cannabis effects commence in a delayed and erratic manner, making it difficult to titrate the required dose. Overdosing and under dosing of medicinal cannabis preparations of unknown THC content were the inevitable consequences often described by physicians of the 19th century. A basic understanding of the pharmacokinetic properties of cannabinoids is necessary to comprehend many issues in context with their medical use, e.g., differences in onset of action and differences in systemic bioavailability between the oral, sublingual and rectal route of administration and inhalation. Of further note in this connection are the teachings contained in “Harm Reduction Associated with Inhalation and Oral Administration of Cannabis and THC”, Grotenhermen, F., J. of Cannabis Therapeutics, Vol. 1 No. 3/4, 2001, pp 133-152—advocating for the most part, abandonment of inhalation and oral administration in favor of rectal, transdermal and sublingual routes.

Aspects of the present invention relate variously to the latter mentioned of the “acceptance” problems associated with dosing of cannabinoid receptor therapeutics—especially in relation to medical uses where the therapeutic impact is especially important, but also in relation to safety of entheogenic or other less formal uses of cannabinoids.

### SUMMARY OF THE INVENTION

Accordingly there remains a problem in the art that is associated with unlocking cannabinoid-related therapies, and which is associated with the reticence of persons skilled in the art and in spite of their acknowledgement of objective evidence of the pharmacological benefits of such therapies: A reticence that is based on their expert medical concerns over the interplay of dose management and its inter-correlated underpinnings of cannabinoid agonist/antagonist potency/stability; and efficacy in its targeted delivery outside of closely-managed treatment settings. That same reticence also drives a regulatory agenda that denies the benefits of otherwise available cannabinoid based therapies.

The present invention addresses this multifaceted problem through a congruent therapeutic modality based on the interrelationship between inhalation delivery of solid lipid particulates carrying lipophilic cannabinoid receptor agonists and/or antagonists, and stability/target potentiation of such agonists/antagonists when carried by solid heterogeneous lipid particles of a homogenate of:
one (or more) lipid(s) whose melting point(s) is (are) substantially above room temperature; in combination with,
one (or more) lipid(s) whose melting point(s) is (are) substantially less than room temperature.

Preferably, this includes solid lipid particle of a homogenate selected from the group comprising:
Solid lipid particle homogenate based on a compounded excipient comprised of a formulation of mutually compatible lipids including a first lipid having a melting point substantially greater than room temperature, and a second lipid having a melting point substantially below room temperature; or,
Solid lipid particle homogenate of lipid phytoextracts fats/oils containing a first lipid having a melting point substantially greater than room temperature, and a second lipid having a melting point substantially below room temperature.
A combination thereof.

It is believed, without wanting to be bound thereby, that both of these types of solid lipid particulate homogenate include crystalized lattice lipids (having the associated higher melting points) and interstitial lipids (having the associated lower melting points), and in which the interstitial lipids interfere with close packing between the crystalized lattice lipids.

In general, the crystalized (“solid” at room temperature) lipids are saturated lipids, and the interstitial (“liquid” at room temperature) lipids are unsaturated lipids; with examples of the former including palmitic acid (m.p. About 63 degrees C.) and stearic acid (m.p. about 70 degrees C.); and, examples of the latter including oleic acid (m.p. about 14 degrees C.) and linoleic acid (m.p. minus 5 degrees C.).

In embodiments of the foregoing wherein the solid lipid particle homogenate is of lipid phytoextracts fats/oils, examples may include solid lipid particle homogenate of extracted cannabis fats/oils or from vegetable oils (e.g. sunflower oil). It is also notable that the solid lipid particle homogenate of extracted cannabis-endogenous fats/oils can include endogenous essential oils that include cannabis phytoterpenoids, such as limonene, myrcene, α-pinene, linalool, β-caryophyllene, caryophyllene oxide, nerolidol and phytol. Cannabis phytoterpenes contribute to the “entourage effect” of Cannabis extracts by synergistically enhancing or modulating the effects of the cannabinoids. Russo, E. B., (2011), Br J Pharmacol. August; 163(7): 1344-1364—and to that extent these are also of particular relevance to the cannabinoid receptor therapies associated with the present invention.

Although inhalation therapies as contemplated herein can extend to vaporization modes, preferred modes include propellant or inspiration of dry solid lipid particulates (e.g. lyophilized homogenate) according to the present invention, or “wet” (e.g. aqueous solution) aerosols of solid lipid particle homogenate. These latter two modes are respectively further preferred (although not necessarily exclusively useful for), pulmonary applications (the dry particulates incur less impingement losses enroute to absorptions sites in the lungs) and nasal applications (the “wet” particles are more disposed to being locally captured at the nasal absorption sites) targeted therapies. Of these latter two modes, wet aerosol therapies are principally preferred owing at least in part to the further complication associated with dry particle adhesion—see e.g. Colloids and Surfaces A: Physicochemical and Engineering Aspects, Volume 165, Issues 1-3,30 May 2000, Pages 3-10, in relation to the adhesion of dry particles in the nanometer to micrometer-size range. This is relevant in that solid lipid particles of homogenates according to the present invention desirably fall within the microparticle and nanoparticle size ranges. Note for reference, that the current IUPAC definition of a microparticle is particle with dimensions between 1×10-7 and 1×10-4 m. The lower limit between micro- and nano-sizing is the subject of a general consensus among the standards groups is that 1-100 nm defines the overall size range of a nanoparticle.

Packaging for homogenates according to this aspect of the present invention advantageously include provisions resisting oxygen and moisture permeability, inert gas headspace flushing and where applicable inert propellants, oxygen scavenging liners or sachets, etc. Especially preferred packages include metered dosing provisions.

Although broader therapeutic applications are contemplated, it is instructive to consider the usefulness of the inhaler packaged solid cannabinoid carrying lipid particle homogenate in its role for treating irritable bowel and similar conditions—which can strike suddenly and unpredictably in the course of a sufferer's day to day activities—and the importance in such a circumstance for a patient to be able to immediately self-administer a reliable dose of effective cannabinoid receptor API.

The present invention also relates, for example, to co-therapeutic uses of cannabinoid receptor agonists/antagonists with opiates—and in particular, with either morphine or methadone—either generally, or in conjunction with the use of cannabinoid receptor active pharmaceutical agents in combination with the above described solid lipid particles, with or without the use of inhalation delivery modalities.

These and other aspects of the present invention are elaborated in the detailed description below, and still others will occur to persons skilled in the art in light thereof.

### DETAILED DESCRIPTION OF THE INVENTION

Aspects of the present invention relate to lipophilic APIs, which are substances used in a pharmaceutical product, intended to furnish pharmacological activity or to otherwise have direct effect in the diagnosis, cure, mitigation, treatment or prevention of disease, or to have direct effect in restoring correcting or modifying physiological functions in mammals. The present invention may in addition, also be employed in connection with lipophilic bioactive nonessential nutritional agents and lipophilic essential nutrients such as required for normal body functioning including certain vitamins, dietary minerals, essential fatty acids and essential amino acids.

Aspects of present invention also relate especially, more particularly to lipophilic APIs which impact on the endocannabinoid system of mammals and in particular, that of humans. The endocannabinoid system is a complex lipid signaling network in which different proteins play distinct roles in the control or modulation of numerous physiological and pathophysiological processes (Pertwee, 2005; Di Marzo, 2008). The system comprises cannabinoid receptors (CB1 and CB2, and orphan receptor GPR55, as well as others). Arachidonic acid-derived ligands also promiscuously target other receptors like, e.g. TRPV1 and PPAR-gamma (O'Sullivan, 2007; De Petrocellis and Di Marzo, 2010; Ross, 2009; Pertwee, 2010). In any case, both cannabinoid receptor agonists and antagonists have therapeutic applications (Di Marzo, 2008; Oesch and Gertsch, 2009; Pertwee, 2009). The importance of the present invention in relation to cannabinoid therapy relates particularly to addressing the problems associated with such therapies, as mentioned previously herein—and as distinguished from other lipophilic API's, lipophilic bioactive nonessential nutrients and lipophilic essential nutrients.

Direct cannabinoid receptor ligands are compounds that show high binding affinities (esp. those in the lower nM size range) for cannabinoid receptors and exert discrete functional effects (e.g. agonism, neutral antagonism or inverse agonism). There are also indirect ligands which target either key proteins within the endocannabinoid system that regulate tissue levels of endocannabinoids or allosteric sites on the CB1 receptor. Certain plant natural products, including some cannabinoids, possess at least some of these properties.

Cannabinoid receptors are located in various mammalian organs and cell types that are associated with the mammalian endocannabinoid system: which in turn is associated in diverse ways, with the physiological processes affecting appetite, pain-sensation, mood, and memory—amongst others. They are generally classed as cellular membrane receptors which fall within the G protein-coupled receptor superfamily. (See, for example: Howlett A C (August 2002), “The cannabinoid receptors” Prostaglandins Other Lipid, Mediat. 68-69: 619-31; and, Mackie K (May 2008), “Cannabinoid receptors: where they are and what they do”. J. Neuroendocrinol. 20 Suppl 1: 10-4; and, Graham E S, Ashton J C, Glass M (2009), “Cannabinoid receptors: a brief history and “what's hot””. Front. Biosci. 14 (14): 944-57.) As such, cannabinoid receptors have been associated with seven transmembrane spanning domains. (see for example: Sylvaine G, Sophie M, Marchand J, Dussossoy D, Carriere D, Carayon P, Monsif B, Shire D, L E Fur G, Casellas P (1995), “Expression of Central and Peripheral Cannabinoid Receptors in Human Immune Tissues and Leukocyte Subpopulations”. Eur J Biochem. 232 (1): 54-61.

Cannabinoid receptors can be activated by three major groups of agonist ligands, for the purposes of the present invention and whether or not explicitly denominated as such herein, lipophilic in nature and classed respectively as: endocannabinoids (produced endogenously by mammalian cells); phyto-cannabinoids (such as cannabidiol, produced by the cannabis plant); and, synthetic cannabinoids (such as HU-210).

The most widely known subtypes of cannabinoid receptors are referenced as CB1 and CB2. (See Matsuda L A, Lolait S J, Brownstein M J, Young A C, Bonner T I (1990), “Structure of a cannabinoid receptor and functional expression of the cloned cDNA”. Nature 346 (6284): 561-4; Gérard C M, Mollereau C, Vassart G, Parmentier M (1991), “Molecular cloning of a human cannabinoid receptor which is also expressed in testis”. Biochem. J. 279 (Pt 1): 129-34. The CB1 receptor is expressed mainly in the brain (central nervous system or “CNS”), but also in the lungs, liver and kidneys, while the CB2 receptor is expressed mainly in the immune system and in hematopoietic cells (see for example, Pacher P, Mechoulam R (2011), “Is lipid signaling through cannabinoid 2 receptors part of a protective system?”. Prog Lipid Res. 50 (2): 193-211.) The protein sequences of CB1 and CB2 receptors are in general about 44% similar, (see for example: Latek, D; Kolinski, M; Ghoshdastider, U; Debinski, A; Bombolewski, R; Plazinska, A; Jozwiak, K; Filipek, S (2011), “Modeling of ligand binding to G protein coupled receptors: Cannabinoid CB1, CB2 and adrenergic β2 AR”. Journal of Molecular Modeling 17 (9): 2353-66; and, Munro S, Thomas K L, Abu-Shaar M (1993). “Molecular characterization of a peripheral receptor for cannabinoids”. Nature 365 (6441): 61-65.) but note too that the respective CB1 and CB2 transmembrane regions of the receptors have amino acid similarities that approximate 68%, (see for example Sylvaine G, Sophie M, Marchand J, Dussossoy D, Carriere D, Carayon P, Monsif B, Shire D, L E Fur G, Casellas P (1995), “Expression of Central and Peripheral Cannabinoid Receptors in Human Immune Tissues and Leukocyte Subpopulations”. Eur J Biochem. 232 (1): 54-61.)

Cannabinoid receptor type 1 (hence the reference: “CB1”) receptors are perhaps among the most widely expressed G protein-coupled receptors in the mammalian brain. This arises out of endocannabinoid-mediated depolarization-induced suppression of inhibition: a notably common form of short-term plasticity in which the depolarization of a single neuron induces a reduction in GABA-mediated neurotransmission. Endocannabinoids (for example) released from the depolarized post-synaptic neuron bind to CB1 receptors in the pre-synaptic neuron and cause a reduction in GABA release. This subtype of receptors are also found in other parts of the body—e.g. in the liver, activation of the CB1 receptors is known to increase de novo lipogenesis, (see for example: Osei-Hyiaman D, DePetrillo M, Pacher P, Liu J, Radaeva S, Bátkai S, Harvey-White J, Mackie K, Offertáler L, Wang L, Kunos G (2005), “Endocannabinoid activation at hepatic CB1 receptors stimulates fatty acid synthesis and contributes to diet-induced obesity”. J. Clin. Invest. 115 (5): 1298-305.

CB2 receptors manifest mainly on T cells of the immune system, on macrophages and B cells, and in hematopoietic cells—although they also perform a function in keratinocytes, and are expressed at peripheral nerve termini. In general, these receptors play a role in antinociception, (pain relief). Accordingly, and although they manifest in mammalian brains, (primarily in association with microglial cells), the dominant efficacy of CB2 receptor-mediated cannabinoid agonists arise out of their impact on the immune and immune-derived cells (e.g. leukocytes, various populations of T and B lymphocytes, monocytes/macrophages, dendritic cells, mast cells, Kupffer cells in the liver, etc.), as well as other cellular targets, including by way of example, endothelial and smooth muscle cells, fibroblasts of various origins, cardiomyocytes, and certain neuronal elements of the peripheral or central nervous systems. (See for example: Pacher P, Mechoulam R (2011), “Is lipid signaling through cannabinoid 2 receptors part of a protective system?”. Prog Lipid Res. 50 (2): 193-211.)

In addition, minor variations of the CB1 and CB2 receptors have been identified. Cannabinoids bind reversibly and stereo-selectively to the cannabinoid receptors. Subtype selective cannabinoids have been developed which may have advantages for treatment of certain diseases such as obesity, (see for example Kyrou I, Valsamakis G, Tsigos C (November 2006), “The endocannabinoid system as a target for the treatment of visceral obesity and metabolic syndrome”. Ann. N. Y. Acad. Sci. 1083: 270-305.)

There is also evidence of non-CB1 and non-CB2 cannabinoid receptors (see for example: Begg M, Pacher P, Bátkai S, Osei-Hyiaman D, Offertáler L, Mo F M, Liu J, Kunos G (2005), “Evidence for novel cannabinoid receptors”. Pharmacol. Ther. 106 (2): 133-45.), which are expressed in endothelial cells as well as in the central nervous system. Ryberg E, Larsson N, Sjögren S, Hjorth S, Hermansson N O, Leonova J, Elebring T, Nilsson K, Drmota T, Greasley P J (2007). “The orphan receptor GPR55 is a novel cannabinoid receptor”. Br. J. Pharmacol. 152 (7): 1092-1101, also describes the binding of several cannabinoids to the G protein-coupled receptor GPR55 in the brain. The existence of additional cannabinoid receptors is further indicated by observations of the incongruous activity of compounds such as abnormal cannabidiol that produce cannabinoid-like effects on blood pressure and inflammation, yet do not activate either CB1 or CB2, (see for example: Járai Z, Wagner J A, Varga K, Lake K D, Compton D R, Martin B R, Zimmer A M, Bonner T I, Buckley N E, Mezey E, Razdan R K, Zimmer A, Kunos G (November 1999), “Cannabinoid-induced mesenteric vasodilation through an endothelial site distinct from CB1 or CB2 receptors”. Proc. Natl. Acad. Sci. U.S.A. 96 (24): 14136-41; and, McHugh D, Tanner C, Mechoulam R, Pertwee R G, Ross R A (February 2008), “Inhibition of human neutrophil chemotaxis by endogenous cannabinoids and phytocannabinoids: evidence for a site distinct from CB1 and CB2”. Mol. Pharmacol. 73 (2): 441-50.) Evidence supports the view that the N-arachidonoyl glycine (NAGly) receptor GPR18 is the molecular identity of the abnormal cannabidiol receptor and additionally suggests that NAGly, the endogenous lipid metabolite of anandamide (also known as arachidonoylethanolamide or AEA), initiates directed microglial migration in the CNS through activation of GPR18, see McHugh D, Hu SS-J, Rimmerman N, Juknat A, Vogel Z, Walker J M, Bradshaw H B (March 2010), “N-arachidonoyl glycine, an abundant endogenous lipid, potently drives directed cellular migration through GPR18, the putative abnormal cannabidiol receptor”. BMC Neuroscience 11: 44. Still other studies support the view that the “orphan” receptor GPR55 should in fact be characterized as a cannabinoid receptor, on the basis of sequence homology at the binding site—and still further studies have revealed that GPR55 does indeed respond to cannabinoid ligands, (see for example: Johns D G, Behm D J, Walker D J, Ao Z, Shapland E M, Daniels D A, Riddick M, Dowell S, Staton P C, Green P, Shabon U, Bao W, Aiyar N, Yue T L, Brown A J, Morrison A D, Douglas S A (November 2007), “The novel endocannabinoid receptor GPR55 is activated by atypical cannabinoids but does not mediate their vasodilator effects”. Br. J. Pharmacol. 152 (5): 825-31). This latter profile suggests a distinct (i.e. non-CB1 and non-CB2) receptor that responds to a variety of both endogenous and exogenous cannabinoid ligands, and supports the categorization of GPR55 as “the CB3 receptor”, (see Overton H A, Babbs A J, Doel S M, Fyfe M C, Gardner L S, Griffin G, Jackson H C, Procter M J, Rasamison C M, Tang-Christensen M, Widdowson P S, Williams G M, Reynet C (March 2006), “Deorphanization of a G protein-coupled receptor for oleoylethanolamide and its use in the discovery of small-molecule hypophagic agents”. Cell Metab. 3 (3): 167-75. Yet other cannabinoid-associated receptor activity has been discovered in the hippocampus, see de Fonseca F R, Schneider M (June 2008), “The endogenous cannabinoid system and drug addiction: 20 years after the discovery of the CB1 receptor” (PDF). Addict Biol 13 (2): 143-6, suggesting that there may be at least two more cannabinoid receptors. GPR119 has also been suggested as a fifth cannabinoid receptor, (See: Brown A J (November 2007) “Novel cannabinoid receptors”. Br. J. Pharmacol. 152 (5): 567-75.)

Aspects of the present invention relate variously to lipophilic cannabinoid receptor ligands (molecules that engage with the active site or such receptors), particularly agonistic and antagonistic ligands (i.e. substances initiating a physiological response through engagement with such a receptor, and especially to lipophilic ligand species.

Cannabinoid receptors are variously activated by lipophilic cannabinoids, generated naturally inside the body (endocannabinoids) or introduced into the body as cannabis or a related synthetic compound. After the receptor is engaged, multiple intracellular signal transduction pathways are activated.

Lipophilic cannabinoids are generally grouped as endocannabinoids (most typically as mammalian endocannabinoids); phytocannabinoids, from plant sources; and synthetic cannabinoids. Such cannabinoids are also often classified into the following subclasses: Cannabigerols (CBG); Cannabichromenes (CBC); Cannabidiol (CBD); Tetrahydrocannabinol (THC); Cannabinol (CBN); Cannabidiol (CBDL); Cannabicyclol (CBL); Cannabielsoin (CBE); and, Cannabitriol (CBT).

Phytocannabinoids are naturally occurring plant compounds found, for example, in the Cannabis sativa plant. Delta-9-tetrahydrocannabinol (Δ9-THC) is the main psychoactive ingredient in cannabis. Cannabidiol (CBD) is another important component, which makes up about 40% of the plant resin extract.

As previously mentioned hereinabove, phytoterpenoids are also significant in their effects in relation to the present invention. Cannabis derived phytoterpenoids according to the present invention, include myrcene, caryophyllene, pinene, terpineol, borneol, linalool, eucalyptol, nerolidol, phellandrene, phytol, humulene, pulegone, bergamotene, farnesene, D3-carene, elemene, fenchol, aromadendrene, bisabolene, as well as others. Cannabis phytoterpenes are associated with the “entourage effect” of Cannabis extract by synergistically enhancing or modulating the effects of the cannabinoids. Russo, E. B.,(2011), Br J Pharmacol. August; 163(7): 1344-1364.

Myrcene is the most common terpene in Cannabis plant strains (up to 60% of the essential oils of certain varieties) and is a potent analgesic, anti-inflammatory and antibiotic. It blocks the action of cytochrome, aflatoxin B, and other pro-mutagenic carcinogens. Cytochrome P450 is a mixed oxidase enzyme primarily responsible for the metabolism of cannabinoids. Inhibition of cytochrome P450 with agents such as mycene, or others such as fluconazole, miconazole or amentoflavone as P450 inhibitors in a cannabinoid SLP formulation can significantly prolong the pharmacological effects of cannabinoids. (Caution may need to be observed here—variations in P450 activity can affect the metabolism and clearance rate of hormones and other drugs leading to potential adverse drug effects). This method offers an advance in the art of pharmaceutical cannabinoids. It also has a relaxing, calming, anti-spasmodic and sedative effect. Acting in synergy with THC, myrcene increases its psychoactive potential—and is therefore useful in offsetting dysphoria in methadone co-therapies according to the present invention.

Limonene is among the next most common of the terpenes found in cannabis resin. Limonene has anti-fungal and anti-bacterial properties and is also anti-carcinogenic. It prevents the deterioration of the RAS gene, one of the factors that contribute to the development of tumors. It also protects against Aspergillus and carcinogens present in smoke. Limonene quickly and easily penetrates the blood-brain barrier, with associated increases in systolic blood pressure. During testing on the effects of limonene, participants experienced an increase in attention, mental focus, well-being and even sex drive. Limonene has been used in spray form, to treat depression and anxiety. It also has the effect of reducing the unpleasantness of gastric acid and stimulates the immune system.

Caryophyllene is a local anti-inflammatory and analgesic, and has the particularity of selectively activate the cannabinoid 2 receptors (CB2), while it is not a cannabinoid.

Pinene is used in medicine as an expectorant, bronchodilator, anti-inflammatory and local antiseptic. It also crosses the hemato encaphalic barrier very easily, where it acts as an inhibitor of acetylcolynesterasics, preventing the destruction of molecules responsible for the transmission of information, which results in memory improvement. Pinene can partially moderate the effects of THC, which leads to a decrease in the acetylcholine levels and THC memory impairment.

Terpineol is associated with the sedative effect of some Cannabis plant strains, and is often found in strains that have a high level of pinenes.

Borneol is both relaxing and psychoactive.

Linalool is currently used in the treatment of various cancers. It also has a powerful calming action, anti-anxiety, and produces a sedative effect. It also has analgesic and anti-epileptic properties.

Eucalyptol (also called 1,8-cineol) relieves pain and improves concentration and inner balance.

Nerolidol, has anti-fungal, anti-leishmaniasis and anti-malarial properties. It also produces a sedative effect.

Other terpenes that can be found in Cannabis plant resin are, for example, phellandrene, phytol, humulene, pulegone, bergamotene, farnesene, D3-carene, elemene, fenchol, aromadendrene, bisabolene, and still others.

Cannabinoids can to some degree, be differentiated on the basis of psychoactive effects: CBG, CBC and CBD are not known to be psychologically active agents; and, THC, CBN and CBDL along with some other cannabinoids are psychoactive to varying degrees. CBD is associated with anti-anxiety effects and possibly counteracting the psychoactive effects of THC (the ratio of CBD to THC in a cannabinoid mixture is relevant—with CBD serving as an antagonist to THC's agonist effect—and the preservation of this relationship is particularly important in metering the respective anti-anxiety vs psychoactive effects of the combined constituency). Note also that when THC is exposed to the air, it becomes oxidized and forms CBN which also interacts with THC to lessen its impact due to the altered CBN:THC ratio.

THC, (as well as two other major endogenous compounds that bind to the cannabinoid receptors—anandamide and 2-arachidonylglycerol), produce most of their effects by binding to both the CB1 and CB2 cannabinoid receptors. While the effects mediated by CB1, mostly in the central nervous system, those mediated through CB2 activation are not equally well defined. Separation between the therapeutically undesirable psychotropic effects, and the clinically desirable ones is to at least some degree possible through the selective use and administration of cannabinoid receptor agonists.

The list of synthetic cannabinoids is extensive, and includes the following:

AM-087 is an analgesic drug that is a cannabinoid agonist derivative of Δ8THC substituted on the 3-position side chain and a potent CB1 agonist; AM-251 is an inverse agonist at the CB1 cannabinoid receptor with close structural similarity to SR141716A (rimonabant), both of which are biarylpyrazole cannabinoid receptor antagonists as well as μ-opioid receptor antagonist; Methanandamide (AM-356) is a stable chiral analog of anandamide and acts on the cannabinoid receptors with a Ki of 17.9 nM at CB1 and 868 nM at CB2; AM-374—palmitylsulfonyl fluoride; AM-381—stearylsulfonyl fluoride; AM404, also known as N-arachidonoylaminophenol, is an active metabolite of paracetamol (acetaminophen) thought to induce its analgesic action through its activity on the endocannabinoid, COX, and TRPV systems, all of which are present in pain and thermoregulatory pathways; AM-411 is an analgesic that is a cannabinoid agonist; AM-411 is a potent and fairly selective CB1 full agonist and produces similar effects to other cannabinoid agonists such as analgesia, sedation, and anxiolysis; AM-630 (6-lodopravadoline) acts as a potent and selective inverse agonist for the cannabinoid receptor CB2, selectivity over CB1 where it acts as a weak partial agonist; AM-661—1-(N-methyl-2-piperidine)methyl-2-methyl-3-(2-iodo)benzoylindole; JWH-018 (1-pentyl-3-(1-naphthoyl)indole) or AM-678 is an analgesic chemical from the naphthoylindole family that acts as a full agonist at both the CB1 and CB2 cannabinoid receptors, with some selectivity for CB2; AM-679 acts as a moderately potent agonist for the cannabinoid receptors; AM-694 (1-(5-fluoropentyl)-3-(2-iodobenzoyl)indole) acts as a potent and selective agonist for the cannabinoid receptor CB1; AM-735—3-bornyl-Δ8-THC, a mixed CB1/CB2 agonist; AM-855 is an analgesic cannabinoid agonist at both CB1 and CB2 with moderate selectivity for CB1; AM-881—a chlorine-substituted stereoisomer of anandamide whose Ki=5.3 nM at CB1 and 95 nM at CB2; AM-883 an allyl-substituted stereoisomer of anandamide whose Ki=9.9 nM at CB1 and 226 nM at CB2; AM-905 is an analgesic cannabinoid which acts as a potent and reasonably selective agonist for the CB1 cannabinoid receptor; AM-906 is an analgesic drug which is a cannabinoid agonist and is a potent and selective agonist for the CB1 cannabinoid receptor; AM-919 is an analgesic cannabinoid receptor agonist, potent with respect to both CB1 and CB2; AM-926—a potent agonist at both CB1 and CB2 with moderate selectivity for CB1; AM-938 is an analgesic drug which is a cannabinoid receptor agonist and while it is still a potent agonist at both CB1 and CB2, it is reasonably selective for CB2; AM-1116—a dimethylated stereoisomer of anandamide; AM-1172—an endocannabinoid analog specifically designed to be a potent and selective inhibitor of AEA uptake that is resistant to FAAH hydrolysis; AM-1220 is a potent and moderately selective agonist for the cannabinoid receptor CB1; AM-1221 acts as a potent and selective agonist for the cannabinoid receptor CB2; AM-1235 (1-(5-fluoropentyl)-3-(naphthalen-1-oyl)-6-nitroindole) acts as a potent and reasonably selective agonist for the cannabinoid receptor CB1; AM-1241 (1-(methylpiperidin-2-ylmethyl)-3-(2-iodo-5-nitrobenzoyl)indole) is a potent and selective agonist for the cannabinoid receptor CB2, with analgesic effects in mammals, particularly against “atypical” pain such as hyperalgesia and allodynia, and has also shown efficacy in the treatment of amyotrophic lateral sclerosis in mammalian models; AM-1248 acts as a moderately potent agonist for both the cannabinoid receptors CB1 and CB2; AM-1710—a CB2 selective cannabilactone with 54x selectivity over CB1; AM-1714 acts as a reasonably selective agonist of the peripheral cannabinoid receptor CB2 and has both analgesic and anti-allodynia effects; AM-2201 (1-(5-fluoropentyl)-3-(1-naphthoyl)indole) acts as a potent but nonselective full agonist for the cannabinoid receptor; AM-2212—a potent agonist at both CB1 and CB2; AM-2213—a potent agonist at both CB1 and CB2; AM-2232 (1-(4-cyanobutyl)-3-(naphthalen-l-oyl)indole) acts as a potent but unselective agonist for the cannabinoid receptors CB1 and CB2; AM-2233 acts as a highly potent full agonist for the cannabinoid receptors CB1 and CB2 and has been found to fully substitute for THC in certain mammalian studies, with a potency lower than that of JWH-018 but higher than WIN 55,212-2; AM-2389 acts as a potent and reasonably selective agonist for the CB1 receptor; AM-3102—an analog of oleoylethanolamide, (the endogenous agonist for proliferator-activated receptor α (PPARα)) it acts as a weak cannabinoid agonist at CB1 and at CB2; AM-4030 an analgesic which is potent agonist at both CB1 and CB2, but also reasonably selective for CB1; AM-4054 is a potent but slow-onset agonist with CB1 affinity and selectivity CB1 over CB2; AM-4113—a CB1 selective neutral antagonist; AM-6545 acts as a peripherally selective silent antagonist for the CB1 and was developed for the treatment of obesity; JWH-007—an analgesic which acts as a cannabinoid agonist at both the CB₁ receptor and CB₂ receptors, with some selectivity for CB₂, JWH-007 is an analgesic which acts as a cannabinoid agonist at both the CB₁ and CB₂ receptors; JWH-015 acts as a subtype-selective cannabinoid agonist which binds almost 28× more strongly to CB₂ than CB₁. and has been shown to have immunomodulatory effects, and may be useful in the treatment of pain and inflammation; JWH-018 an analgesic which acts as a full agonist at both the CB₁ and CB₂ cannabinoid receptors and produces effects similar to those of THC; JWH-019—an agonist at both CB₁ and CB₂ receptors and is an analgesic from the naphthoylindole family that acts as a cannabinoid agonist at both the CB₁ and CB₂ receptors; JWH-030—an analgesic which is a partial agonist at CB₁ receptors; JWH-047—a potent and selective agonist for the CB₂ receptor, JWH-048—a potent and selective agonist for the CB₂ receptor, JWH-051—an analgesic with a high affinity for the CB₁ receptor, but is a much stronger agonist for CB₂, JWH-057—a 1-deoxy analog of Δ8-THC that has very high affinity for the CB₂ receptor, but also has high affinity for the CB₁ receptor; JWH-073—an analgesic which acts as a cannabinoid agonist at both the CB₁ and CB₂ receptors. It is somewhat selective for the CB₁ subtype; JWH-081—an analgesic which acts as an agonist at both the cannabinoid CB1 AND CB2 receptors; JWH-098—a potent and fairly selective CB₂ agonist; JWH-116—a CB₁ ligand; JWH-120—a potent and 173-fold selective CB₂ agonist; JWH-122—a potent and fairly selective CB₁ agonist; JWH-133—a potent and highly selective CB₂ receptor agonist; ¹JWH-139—3-(1,1-dimethylpropyl)-6,6,9-trimethyl-6a,7,10,10a-tetrahydro-6H-benzo[c]chromene; JWH-147—an analgesic from the naphthoylpyrrole family, which acts as a cannabinoid agonist at both the CB₁ and CB₂ receptors; JWH-148—a moderately selective ligand for the CB₂ receptor, with more than 8 times selectivity over the CB₁ subtype; JWH-149—a potent and fairly selective CB₂ agonist; JWH-161—a CB1 ligand; JWH-164—a potent cannabinoid agonist; JWH-166—a potent and highly selective CB₂ agonist; JWH-167—a weak cannabinoid agonist from the phenylacetylindole family; JWH-171—an analgesic which acts as a cannabinoid receptor agonist; JWH-175—(1-pentylindol-3-yl)naphthalen-1-ylmethane, 22 nM at CB₁, JWH-176—1-([(1E)-3-pentylinden-1-ylidine]methyl)naphthalene; JWH-181—a potent cannabinoid agonist; JWH-182—a potent cannabinoid agonist with some selectivity for CB₁; JWH-184—1-pentyl-1H-indol-3-yl-(4-methyl-1-naphthyl)methane; JWH-185—1-pentyl-1H-indol-3-yl-(4-methoxy-1-naphthyl)methane; JWH-192—(1-(2-morpholin-4-ylethyl)indol-3-yl)-4-methylnaphthalen-1-ylmethane; JWH-193—(1-(2-morpholin-4-ylethyl)indol-3-yl)-4-methylnaphthalen-1-ylmethanone; JWH-194—2-methyl-1-pentyl-1H-indol-3-yl-(4-methyl-1-naphthyl)methane; JWH-195—(1-(2-morpholin-4-ylethyl)indol-3-yl)-naphthalen-1-ylmethane; JWH-196—2-methyl-3-(1-naphthalenylmethyl)-1-pentyl-1H-Indole; JWH-197—2-methyl-1-pentyl-1H-indol-3-yl-(4-methoxy-1-naphthyl)methane; JWH-198—(1-(2-morpholin-4-ylethyl)indol-3-yl)-4-methoxynaphthalen-1-ylmethanone; JWH-199—(1-(2-morpholin-4-ylethyl)indol-3-yl)-4-methoxynaphthalen-1-ylmethane; JWH-200—an analgesic from the aminoalkylindole family, which acts as a cannabinoid receptor agonist; JWH-203—an analgesic from the phenylacetylindole family, which acts as a cannabinoid agonist with approximately equal affinity at both the CB₁ and CB₂ receptors; JWH-205—142-methyl-1-pentylindol-3-yl)-2-phenylethanone; JWH-210—an analgesic from the naphthoylindole family, which acts as a potent cannabinoid agonist at both the CB₁ and CB₂ receptors; JWH-213—a potent and fairly selective CB₂ agonist; JWH-229—1-methoxy-3-(1′,1′-dimethylhexyl)-Δ⁸-THC, a dibenzopyran cannabinoid which is a potent CB₂ agonist; JWH-234—a cannabinoid agonist with selectivity for CB₂; JWH-250—an analgesic from the phenylacetylindole family, which acts as a cannabinoid agonist at both the CB₁ and CB₂ receptors; JWH-251—(1-pentyl-3-(2-methylphenylacetyl)indole); JWH-258—a potent and mildly selective CB₁ agonist; JWH-302—(1-pentyl-3-(3-methoxyphenylacetyl)indole); JWH-307—an analgesic from the naphthoylpyrrole family, which acts as a cannabinoid agonist at both the CB₁ and CB₂ receptors that is somewhat selective for the CB₂ subtype; JWH-350—a 11-nor-1-methoxy-3-(1′,1′-dimethylheptyl)-9α-hydroxyhexahydrocannabinol has a 33-fold selectivity for the CB₂ receptor and high CB₂receptor affinity with little affinity for the CB₁ receptor; JWH-359—a dibenzopyran cannabinoid that is a potent and selective CB₂ receptor agonist; JWH-387—1-pentyl-3-(4-bromo-1-naphthoyl)indole, an analgesic from the naphthoylindole family, which acts as a potent cannabinoid agonist at both receptors CB₁ and CB₂; JWH-398—an analgesic chemical from the naphthoylindole family, which acts as a potent cannabinoid agonist at both receptors with a Kᵢ of 2.3 nM at CB₁ and 2.8 nM at CB₂; JWH-424—a potent and moderately selective CB₂ agonist with a Kᵢ of 5.44 nM at CB₂ and 20.9 nM at CB₁; HU-210 is a cannabinoid that is 100 to 800 times more potent than natural THC from cannabis and has an extended duration of action and is a ponntent analgesic with many of the same effects as natural THC; Ajulemic acid (AB-III-56, HU-239, IP-751, CPL 7075, CT-3, Resunab) is a cannabinoid derivative of the non-psychoactive THC metabolite 11-nor-9-carboxy-THC that shows useful analgesic and anti-inflammatory effects without causing a subjective “high”. It is being developed for the treatment of neuropathic pain and inflammatory conditions such as arthritis and for the treatment of orphan life-threatening inflammatory diseases; HU-243 (AM-4056) is a cannabinoid which is a potent agonist at both the CB₁ and CB₂ receptors; HU-308 acts as a cannabinoid agonist and is highly selective for the CB₂ receptor subtype. It has analgesic effects, promotes proliferation of neural stem cells, and protects both liver and blood vessel tissues against oxidative stress via inhibition of TNF-α; HU-331 is a quinone anticarcinogenic synthesized from cannabidiol; HU-336 is a strongly antiangiogenic compound, it inhibits angiogenesis by directly inducing apoptosis of vascular endothelial cells without changing the expression of pro- and anti-angiogenic cytokines and their receptors; HU-345 (cannabinol quinone) is a drug that is able to inhibit aortic ring angiogenesis more potently than its parent compound cannabinol; CP 47,497 or (C7)-CP 47,497 is a cannabinoid receptor agonist drug, developed by Pfizer in the 1980s. It has analgesic effects and is a potent CB₁ agonist.

Notwithstanding the therapeutic potential of cannabinoid receptor therapies, the reasons for the decline in medical use of cannabinoids often have to do with variable potency; instability; unpredictability of response by oral route; imprecise dosing (lack of clarity). In a preferred aspect of the present invention, high proportions of hydrophobic cannabinoids are carried in chemically and physically stable solid lipid particles (SLP), to be dispensed intra nasally by (preferably metered) dose inhalers (MDI). These microparticles and nanoparticles reach deep into the nasal cavity where they are readily dispersed in the mucous membrane and rapidly absorbed through the nasal epithelium into blood, plasma and tissue. When cannabinoids are packaged in a familiar medical device that dispenses precise doses of known potency and duration, the negative images most Physicians associate with cannabinoids are addressed. This results in improved treatment of intractable Irritable Bowel Syndrome (IBS), Crohn's Disease (CD) and Ulcerative Colitis (UC), chronic, debilitating medical conditions for which no other drug or combination of drugs has proven to be as effective.

In accordance with certain aspects of the present invention cannabinoids are homogenized into stabilized Solid Lipid Particles (SLP) formulated for dosing for example through intra nasally or by pulmonary inhalation. In general, stabilized cannabinoid SLP according to the present invention may also be dosed intravenously, intrathecally, orally, ocularly, trans-dermally and rectally—and in any case such cannabinoid SLP's offer improved delivery to target organs, more rapid dissolution, improved absorption, bioavailability and higher plasma levels. Nevertheless, the inhalation route offers advantages congruent with the metered dosing thereof, that address concerns of the medical community while making cannabinoid therapy practicable for real world patients.

An inhaler is a medical device that delivers a specific amount of API into the nasal cavity by self-administration. The administration of cannabinoids by inhaler in accordance with the present invention is proposed to improve dose delivery, rate of dissolution, absorption and bioavailability of cannabinoid formulations. More particularly, there is a need to provide a dispensable form of cannabinoids suited to these purposes and preferably through the use of multi-dose inhalers (MDI) comprised of a manually operated pump which disperses a stabilized colloidal dispersion of cannabinoid nanoparticles into the nasal cavity. Nasal inhalers require a measured dose be made ready for the patient to dispense. Typically, the liquid dose dispensed by an MDI is less than 1/100 ml to contain the delivered dose of cannabinoid SLP with the nasal cavity. A mode of dispensing highly potent API and in particular cannabinoid agonists/antagonists, which can deliver 1/100 ml doses would be a valuable tool the pharmaceutical field.

In accordance therefor with a related aspect of the present invention, there is provided an inhaler-delivery-device-packaged homogenate of solid heterogeneous-lipid particulates carrying lipophilic cannabinoid receptor agonists and/or antagonists, wherein the solid heterogeneous-lipid particles comprise: one (or more) lipid(s) whose melting point(s) is (are) substantially above room temperature; in combination with, one (or more) lipid(s) whose melting point(s) is (are) substantially less than room temperature. The result is a reliable medical inhaler that can deliver a narrowly targeted size of micro and nano cannabinoid into deep within the nasal cavity. Over 80% of nasally administered cannabinoid formulation can be reasonably expected to be delivered to nasal epithelium where it rapidly disperses and is absorbed, with peak plasma levels achieved, for example, in 7 minutes, (near equivalent to intravenous administration). Cannabinoids delivered by conventional medical inhalers that dispense precise doses of known potency and duration will address prejudices held by physicians who resist prescribing medical marijuana. This will result in increased use of cannabinoid to treat, for example, Irritable Bowel Syndrome and other debilitating GI disease and inflammatory conditions. Inhaler dispensed cannabinoids are also effective in control of spasm, fasciculation's and neurogenic pain of Multiple Sclerosis, chemotherapy induced nausea, radiation induced colitis, controlling terminal cancer break through pain and as a systemic anti-tumorigenic, anti-metastatic cannabinoid agent for treatment of prostate, colon and breast cancer and as a systemic adjunct to topical application in treatment of skin cancers, including melanoma.

A preferred inhaler-delivery-device-packaged homogenate according to this same aspect of the invention, comprises a solid lipid particle of a homogenate selected from the group comprising: Solid lipid particle homogenate based on a compounded excipient comprised of a formulation of mutually compatible lipids including a first lipid having a melting point substantially greater than room temperature, and a second lipid having a melting point substantially below room temperature; or, Solid lipid particle homogenate of lipid phytoextracts fats/oils containing a first lipid having a melting point substantially greater than room temperature, and a second lipid having a melting point substantially below room temperature; or, A combination thereof.

Cannabis extracts where prepared for this (and other aspects of the present invention, wherby Cannabis chemovars high in THC (Tetra-hydro cannabinol), CBD (cannabidiol) and CBV (cannabivardin) were grown, harvested, dried to 20% moisture, ground, sieved to <3 mm and stored at −20oC in the absence of light. Ground plant material was then heated to 113oC for 90 minutes to decarboxylate the plant acids. Although subcritical extraction can be employed, super-critical CO2 extraction was done at packing densities of 0.25 to 0.35 with CO2 pressure at 70 bar and 31oC for 4 to 8 hours. Depressurization removed the CO2 which was scrubbed and recycled for subsequent use. The crude extract was stored under nitrogen at −15oC.

In some embodiments this crude extract was further treated to an ethanolic extraction (2:1 wt:wt) of the crude extract can be done (e.g. at 30oC and then refrigerated at −25oC for 48 hours to precipitate plant waxes and cold filtered with 20 μm filter paper to remove the insoluble fraction The ethanol was removed by vacuum evaporation at 62oC and 172 mBar vacuum, then the vacuum was increased to 50 mBar to remove any residual water. This process yielded a dry phyto-cannabinoid extract which is stored at −25oC in darkness, under nitrogen until required. This ethanol extraction cold filtering step is not employed in the event that cannabinoid extract is to be homogenized directly into a cannabis extract SLP in which the plant waxes assist in SLP formation. (Note in this connection, that in embodiments wherein the solid lipid particle lipids are to be sourced from the above mentioned crude extract directly, this ethanolic step is not performed.)

In embodiments, such as the stearic:sunflower oil combination described herein, the high THC chemovar extract (with ethanol extraction) contained 55-63% THC (tetra-hydro-cannabinol), 1-3% CBD (cannabidiol) and 3-5% other cannabinoids with average total yields of 8.6% based on dry plant weight. High CBD chemovar extract yielded 48-56% CBD, 2-4% THC, 3-5% other cannabinoids with a total yield of 8.5% based on dry plant weight. High CBV (cannabivardin) chemovar extract yielded 38% CBV and 2-4% other cannabinoids with a total yield of 5.8% based on dry plant weight. If desired, the further separation of the extract into pure cannabinoids was accomplished by conventional high pressure liquid chromatography.

Following cannabinoid synthesis and/or extraction and clean-up of a phyto-cannabinoid extract, a method was developed to encapsulate cannabinoids in a Solid Lipid Particle (SLP) excipient matrix.

Phyto-cannabinoid extract and/or synthetic cannabinoid is warmed, dispersed, stirred and dissolved in suitable lipid(s), a warmed antioxidant is added and stirred, followed by addition of an aqueous solution of surfactant. This heated pre-emulsion is then subjected to high pressure homogenization for an appropriate period and the resultant homogenate cooled to room temperature which results in the formation of cannabinoid SLP that vary in size from nano to micro depending on process conditions.

More specifically, To accomplish encapsulation, the 70:30 mixture of solid and liquid lipids were heated to 73° C. to liquefy the solid stearic acid and the heated mixture of lipids was stirred for 10 minutes to achieve uniformity. Under modified nitrogen atmosphere, synthetic cannabinoid or phyto-cannabinoid extract was warmed to the same temperature as mixed lipids and slowly added to the heated lipids while being constantly stirred. A 73° C. aqueous solution of Polysorbate 80 was then slowly added to the heated cannabinoid-lipid mixture while still being constantly stirred (at 20,000 rpm in a Silverston-type mixer for 10 minutes), to achieve a uniform pre-emulsion or pre-homogentate. The high internal stability of cannabinoid SLP's was ensured by selection of chemically compatible components and surfactants (such as the above mentioned Polysorbate 80 or tween 80 or others, e.g. polaxmers) which formed a stable monolayer around each SLP, (to resist coalescence or flocculation).The 73° C. pre-emulsion was placed in a high pressure homogenizer and homogenized for a suitable period to yield a heated (an 83 degree C.—due to heat rise associated with passage through the homogenizer) stabilized oil-water homogenate. The homogenate was permitted to cool to room temperature resulting in the generation of micrometer or nanometer cannabinoid SLP. Initial experiments with lipophillic non cannabinoid essential nutrients also demonstrated that persons skilled in the art, by varying process conditions in accordance with the present invention, could produce SLP sizes ranging from nano-meter to micro-meter diameters while retaining tight particle size distributions. formed into solid lipid particles <100 μm with stable internal structure and an absence of lipid crystallization that can readily form fine aerosol that disperse rapidly in aqueous media like nasal and pulmonary epithelium and are rapidly absorbed by blood, plasma or tissue. Observed particle size distributions in one case were generally between 1 and 8 microns with average size of 5 microns; and another exhibited particle size distribution generally between 14 and 30 microns with average particle size of 23 microns. Other solid lipid particles were produced between 3 and 12 microns in diameter, with average size of 5 microns, for delivery of Cannabinoid SLP to distal alveoli of the pulmonary system and minimize oral and upper airway impingement. In another case, solid lipid particles are produced between 12 and 30 microns in diameter, with average size of 23 microns, to also deliver Cannabinoid SLP or other API-SLP to deep within the nasal and para nasal cavities with significantly diminished carryover to the pulmonary system. In a preferred embodiment, an intra-nasal delivery introduces 5-10 nano meter cannabinoid SLP into the nasal cavities, wherein virtually all of such particles tend to be absorbed by the nasal epithelial. Also, a plume of cannabinoid-α-tocopherol SLP emanating from a pulmonary inhaler exhibited an average particle size distribution of 5 microns, a suitable size range for pulmonary inhalation to maximize delivery to distal alveoli of the pulmonary system and minimize oral cavity and nasopharyngeal impingement. Nuclear magnetic resonance (NMR) was used to determine the size and qualitative nature of the nanoparticles. The selectivity afforded by chemical shift provides information on the physicochemical status of components within the nanoparticles. Scanning electron microscopy (SEM) also provides a way to directly observe and physically characterize nano particles. One must be cognizant of the statistically small sample size and the effect of high vacuum on some nano particles when interpreting these observations. Cannabinoid SLP dispersions in accordance with the practice of the present invention can be produced so as to fall within a reasonably narrow size range.

In a preferred product and method according to the invention, the solid-lipid particles with stable internal structure in which the lower mp lipid is generally interspersed between the crystalline structure of the higher mp lipid. Cannabinoid SLP exhibit stable particle structure with no segregation, leakage, hydrolysis or oxidation of Active Pharmaceutical Ingredient (API) for one year. This process has no known scale-up problems and it was used to produce 1 Kg of SLP comprising 4% excipient lipids:1% α-tocopherol:95% cannabinoid was manufactured in the aforementioned manner. increased surface area generated by formation of very small cannabinoid-α-tocopherol solid lipid particles results in an increased rate of dissolution of cannabinoid-α-tocopherol in nasal and pulmonary epithelium and a concomitant increase in absorption and ultimate concentration of cannabinoid in blood and plasma Note that encapsulation in such a lipid matrix confers a degree of protection against oxidative degradation of THC and other similarly sensitive API. Following twelve months storage at −10oC in darkness, 5 ml screw top bottles containing THC, THC-SLP and THC-α-tocopherol-SLP were extracted in hexane. Extract was concentrated by vacuum evaporation and percent of THC degradation quantitated by gas chromatograpy. 22.6% of THC degraded to Cannabinol; 14.0% of THC-SLP degraded to Cannabinol; 2.1% of THC-α-tocopherol-SLP degraded to Cannabinol. Thus THC-SLP encapsulation stabilized 92% of THC; SLP encapsulation with addition of α-tocopherol stabilized 97.9% of THC. Such a method will offer a significant advance for pharmaceutical cannabinoids.

In preferred combinations hereof, the inhaler-delivery-device-packaged homogenate includes a first lipid which comprises one or more saturated fatty acid(s), and said second lipid comprises one or more unsaturated fatty acid(s). Examples include first lipids such as palmitic acid and stearic acid. Many such lipids are solids at normal body temperature—as well as being biocompatible, biodegradable, and “Generally Recognized As Safe” (GRAS) and available in high purity for a minimal cost. Stearic acid:sunflower oil (70:30) stabilized with an aqueous surfactant solution was finally selected for the preferred manufacture of cannabinoid SLP. Stearic acid was chosen because it is neutral with respect to cholesterol in human blood.

In connection with the inhaler-delivery-device-packaged homogenate as well as other aspect of the present invention, the second lipid includes one or more of the group of saturated fatty acids comprising for example oleic acid and linoleic acid.

The inhaler-delivery-device-packaged solid lipid particle homogenate can be of lipid phytoextracts fats/oils—as in the case wherein the solid lipid particle homogenate of lipid phytoextracts fats/oils comprises one or more of the group selected from solid lipid particle homogenate of extracted cannabis fats/oils; or, solid lipid particle homogenate of one or more vegetable oils. In particular, the inhaler-delivery-device-packaged homogenate can includes a solid lipid particle homogenate of extracted cannabis-endogenous fats/oils, and which advantageously further comprises cannabis-endogenous essential oils. Such essential oils are cannabis phytoterpenoids, and include one or more of the group selected from limonene, myrcene, α-pinene, linalool, β-caryophyllene, caryophyllene oxide, nerolidol and phytol. β-caryophyllene, for example, is an FDA approved food additive, and also present in phyto-cannabinoid extracts of Cannabis and is a selective CB2 cannabinoid receptor agonist. Addition of β-caryophyllene to cannabidiol (CBD) rich phyto-cannabinoid extract increases the agonist effect of CBD on CB2 receptors which are found throughout the gut lining and act to modulate and regulate gastro-intestinal function in normal states and pathological states such as IBS. This finding that will offer a significant advance in the pharmaceutical art of cannabinoids.

Various aspects of the inhaler-delivery-device-packaged homogenate combination according to the present invention can include propellant or inspiration of dry solid lipid particulate homogenate devices; or “wet” pumped aerosols of solid lipid particle homogenate devices. In this latter respect, and to circumvent the aggregation problem sometimes encountered with lyophilized or otherwise dried nanoparticles, it was found to be advantageous to formulate cannabinoid SLP as a stabilized colloidal dispersion of nanoparticles and/or microparticles in a liquid carrier fluid such as water containing appropriate colloidal stabilizers. This liquid carrier can then be filled into, (for example), an MDI inhaler dosing device that employs a manually actuated pump to deliver a precise dose of cannabinoid nanoparticle suspension, intra nasally. Nano particle size distribution remains stable over long periods of time during device storage as the sedimentation of nanoparticles is minimized by selection of appropriate colloidal stabilizers and aqueous phase thickeners in the API formulation. The API in this context is hydrolytically stable and chemically compatible with colloid stabilizers and thickeners and offers an alternative method of choice for delivery of intra nasal nano cannabinoids by Metered Dose Inhalers (MDI). The cannabinoid SLP is formulated with a viscosity thickening agent and colloidal stabilizer and packaged into MDI inhalers under GLP/GMP conditions. The nanoparticle size distribution remains stable over long periods of time during device storage as the sedimentation of nanoparticles and/or microparticles is minimized by selection of appropriate colloidal stabilizers and aqueous phase thickeners in the formulation. Examples of viscosity thickening agents for nano cannabinoids include water soluble polymers like PEG, chitosan, locust bean gum, xanthan gum, carbopol and hydroxyl methyl cellulose. The selected viscosity thickening agent should be biocompatible, water soluble and GRAS for nasal administration. Examples of colloid stabilizer for nano cannabinoids are polyaxmers, Pluronic F127, Tween 20, Tween 80 and salts of fatty acids like sodium stearate which can be a non-ionic or ionic surfactant that is GRAS for nasal use. The monolayer of surfactant that surrounds the API within an SLP also reduces surface tension in an aqueous environment, such as that which surrounds nasal epithelial cell walls. This assists small lipophilic API such as cannabinoid SLP to disperse more readily, increasing the rate and amount of API absorbed in blood. The combination of SLP surfactant monolayer and nasal cell wall phospholipids provided an unexpected improvement in rate of dispersion and absorption of cannabinoids through nasal epithelium into blood. Still in accordance with this aspect of the present invention, nanometer particulates of cannabinoid SLP have been stabilized as a colloidal dispersion in an aqueous carrier fluid containing colloidal stabilizers. This liquid phase can then be delivered as metered volumes using a manual pump to deliver a precise dose of cannabinoid nanoparticle suspension intra nasally. Moreover, following 12 months storage at room temperature, packaged THC was tested for degradation. Gas chromatographic analysis indicated <2% of packaged THC had degraded to cannabinol, indicating the inhaler packaged THC had at least a one year shelf life. Such a method would be a valuable addition to the pharmaceutical packaging art of cannabinoids.

Nasal delivery offers various advantages. No other body aperture provides such uncomplicated access for a patient and offers such patient comfort for delivery. The thin epithelial layer covering the dense vascular bed of the nasal cavity offers rapid absorption, improved delivery, ease of self-titration and an onset rivalling intravenous. Cannabinoids are potent drugs and cannabinoid SLP are 96% cannabinoid, 4% lipid. The nasal and para nasal cavities should capture virtually 100% of 14 to 30 micron sized cannabinoid SLP, retaining the entire dose. 10 mg THC SLP administered by DPI achieved peak plasma level in 7 minutes. The peak plasma level indicated 98% of the administered dose was deposited in the nasal cavity with the balance lost due to inefficiency in nasal inhalation. A “transcribial route” for administration of small cannabinoid SLP transported directly to the brain, offers the potential for strong, prolonged effects after a single small dose, provided however that delivered particles of cannabinoid SLP are in the range of 3 to 5 microns reach the olfactory area of the nasal cavity by nasal inhalation.

In another aspect of the present invention, (one not necessarily tied to either inhalation therapy, or indeed to cannabinoid therapies at all), there is provided a lipophilic active pharmaceutical ingredient, lipophilic bioactive nonessential nutrient agent or lipophilic essential nutrient, in combination with a carrier comprised of mutually compatible lipids including a first crystalized lipid providing a crystalline structure with a second lipid interstitially disposed within that structure and in close-packing-interfering relation with first lipid crystals thereof, and wherein the first lipid is a solid at room temperature, and the second lipid is a liquid at a temperature of about 21 degrees centigrade. Preferrably, the first lipid has a melting point higher than normal internal human body temperature (but as persons skilled in the art will appreciate, must be low enough so that its melting does not result in substantial damage to a temperature labile API. Preferrably, the first lipid is stearic acid and the second lipid is sunflower oil, and the ratio of the first lipid to the second lipid is about 70 parts to 30 parts by weight.

The addition of an antioxidant is preferred: as for example, by way of the addition of alpha tocopherol.

As noted previously in other respects, it is preferred that the combination be in an aqueous excipient-in-water emulsion including a surfactant—with polysorbate surfactant emulsions being exemplary in this connection.

Aspect of this combination according to the present invention relate to a pre-homogenate aqueous, emulsifier-stabilized, uniform emulsion of liquid phase first and second excipient lipids as well as to a homogenate of said pre-homogenate aqueous emulsion and particularly wherein at a temperature below the melting point of at least the first lipid, the homogenate is formed of solid lipid particulates of excipient-borne active pharmaceutical ingredient. Such solid lipid particulates of excipient-borne active pharmaceutical ingredient preferable includes a substantial proportion of numbers of particles in the micrometer and/or nanometer size ranges.

In one form of the invention, such particulates form a dry, friable powder—typically following lyophilzation or the like.

The combinations hereof typically comprise a lipophilic active pharmaceutical ingredient, lipophilic bioactive nonessential nutrient agent or lipophilic essential nutrient, and preferably in an amount comprising 30 to 96% by weight of said combination.

Packaged product combinations hereof can include packaging selected from one of the group selected from dry dispense packaging; wet pump dispense packaging, blister packaging; gel cap dispensing. There are however, advantages to combinations wherein the package is a metered dose dispenser and especially an intra nasal dispenser. In any case, many API's contain oxygen sensitive and light sensitive materials with reactive chemical sites that cause them to degrade in the presence of oxygen, and/or light. Limiting exposure to oxygen, and light can protect sensitive compounds from degradation, extending shelf-life from weeks to years. Oxygen contamination can occurs in pharmaceutical packaging when: the API is exposed to oxygen/light during packaging, when the API remains exposed to oxygen/light in the final package or when oxygen/light permeates through the package and degrades the API. Polymeric oxygen scavengers are employed on the interior surface of semi-permeable containers and can scavenges oxygen from semi-permeable sealed packages, extending the shelf-life of sensitive API in semi-permeable sealed packages to scavenge oxygen from the packages interior. In particular the API could be delta-9-tetrahydrocannabinol, a chemically unstable cannabinoid that rapidly oxidizes to cannabinol (CBN) when exposed to oxygen and light. Other cannabinoids, their synthetic analogues, CB1 and CB2 receptor agonists and antagonists are also known to be subject to degradation. Packaging preferably therefor includes at least one oxygen scavenging element disposed within an oxygen impermeable container. As used herein, the term “oxygen scavenging element” refers to any substance that consumes, depletes or reduces the amount of oxygen from a given environment without negatively affecting the cannabinoid product. Suitable oxygen scavenging elements are known to those skilled in the art. Non-limiting examples of oxygen scavenging elements include, but are not limited to, compositions comprising metal particulates reactive with oxygen like transition metals from the first, second or third transition series of the periodic table: Manganese II or III, Iron II or III, Cobalt II or III, Nickel II or III, Copper I or II, Rhodium II, III or IV, and Ruthenium. The oxygen scavenging transition metal is preferably Iron, Nickel or Copper. The purpose of the oxygen scavenger element is to remove oxygen from within an oxygen impermeable container without negatively affecting the packaged cannabinoid product. In a preferred aspect of the present invention a manually actuated nasal inhaler comprising a container which may be of any shape or size suitable but preferably ergonometrically suited for use by persons suffering from neurodegenerative or chronic debilitating disease to assist and enable them to consistently intra nasal doses of the packaged API without assistance. Packages comprising impermeable containers are also preferably of a suitable interior shape and size to be readily purged of head space gases and of sufficient size to contain about 100 doses of API. Possibly a second oxygen scavenger can also be employed within the container. Such a second oxygen scavenging element can be fused to the inner wall of impermeable container. Oxidizable, organic polymer oxygen scavengers are known in the food packaging art and include substituted or unsubstituted ethylenically unsaturated hydrocarbons and mixtures thereof like polybutadiene, polyisoprene, and styrene-butadiene block copolymer, or polyterpenes such as poly meta-xylenediamine-adipic acid, or acrylates such as polyethylene-methylacrylate-benzyl acrylate.

Packaging issues aside, combinations of the invention according to this aspect can include one or more of the group selected from the lipophilic vitamins; opiates, endogenous cannabinoids, synthetic cannabinoids, solvent extracted (eg especially ethanol extracted) phytocannabinoids from (e.g. and preferably cannabis via carbon dioxide extracted) plant extracts, essential oils of cannabis plant cannabinoids and/or terpenoids, a cannabinoid receptor agonist, and cannabinoid receptor antagonist.

In the case of an opiate, notable examples include methadone or morphine and especially in relation to combinations therapies with with a phyto-cannabinoid extract (predominantly containing a THC/cannabidiol combination with minor proportions of other phyto-canabinoids and/or phyto-terpenoids or synthetic equivalents thereof. This is believed to be associated with an “entourage effect” is the sum of/between multiple synergies), wherein the proportion of opiate is a moderated dose in proportion to a moderating effect of the phyto-cannabinoid extract. (subclinical opiate doses).

The selected API preferably comprises 30 to 96% by weight of the API and lipid excipient combination.

In another aspect of the present invention, there is provided a method for producing a solid lipid particle pre-homogenate, comprising:

a. heating a mixture comprising:
i. a heterogeneous lipid combination including:
   1. one (or more) lipid(s) whose melting point(s) is (are) substantially above room temperature; in combination with,
   2. one (or more) lipid(s) whose melting point(s) is (are) substantially less than room temperature, and
ii. one or more of a group selected from lipophilic API, lipophilic bioactive nonessential nutrient, or lipophilic essential nutrient

b. to above the melting point which is substantially above room temperature sufficient to melt said lipids and reduce the mixtures viscosity; and

c. pre-homogenizing the heated mixture to produce a stable pre-homogenate.

The method of this aspect of the invention further contemplates the addition of surfactant stabilizer to the mixture. The surfactant is preferably a non-ionic surfactant, preferably selected from the group consisting of polysorbates or poloaxmers.

The mixing is preferably carried out for about 10 minutes at about 20,000 rpm, (in for example a Silverton mixer).

Methods according to the present invention also include preparing a solid lipid particle homogenate by heating/homogenizing the heated pre-homogenate mentioned above at about 500 to 1500 bar at least once and preferably twice to produce a further heated (typically with about a further 10 degree C. rise in temperature) homogenate, and then cooling the heated homogenate to about room temperature, to produce a solid lipid homogenate.

The homogenization of the pre-homogenate is carried out to produce solid lipid microparticles and/or nanoparticles in said room temperature homogenate. Note that the pre-homogenization and homogenization are carried out at temperatures above the melting point of the described lipids and is similar to the homogenization of an emulsion. The pre homogenate of the drug loaded lipid melt and the aqueous emulsifier phase (which are added to one another at the same temperature) is obtained by use of a high shear Silversion homogenizer (20,000 rpm for 8 to 10 minutes)—and the quality of the pre-homogenate affects the quality of the final product, hence it is desirable to obtain droplets of only a few micrometers in size. High pressure homogenization of the pre-emulsion is done above the lipid melting point. Smaller particle sizes were obtained at higher processing temperatures because of the lowered viscosity of the lipid phase although higher temperatures can accelerate cannabinoid and lipid carrier degradation. Superior product was obtained with multiple passes through the high-pressure homogenizer, however two homogenization cycles at 500-1500 bar was generally sufficient to produce particles less than 100 nano meters and increasing the number of homogenization cycles ran a risk of actually increasing particle sizes due to particle coalescence.

When desired the method can further include lyophilizing or spray drying of the solid lipid particles.

In one aspect of the present method, the mixture comprises a cannabis carbon dioxide extract wherein said heterogeneous lipids are comprised of cannabis fats and oils from said cannabis extract and particularly in instances where the selected API comprises a carbon dioxide cannabis extract, containing cannabis extracted phytocannabinoids.

In particular, this aspect of the invention relates to cannabis extracted phytocannabinoids is a carbon dioxide cannabis extract residual following ethanolic extraction thereof, and said heterogeneous lipid combination is comprised of lipids from sources other than cannabis.

Other methods may be employed to produce nanoparticles containing cannabinoids. These include:

A) Hot-melt-chill process: This process requires and API with a melting point below around 100C and waxy or lipidic excipients that also melt below around 100C, and in which the API is soluble in both the molten and solid state, or at least does not recrystallize from the solid state on cooling. In this process, API and the waxy or lipidic excipients that are compatible with the API are heated to melting, and well mixed. This mixture is then emulsified under high shear mixing into a hot aqueous solution of pharmaceutically appropriate emulsifiers (hotter than the melting point of the API/lipidic mixture) to form a pre-emulsion of API/lipid droplets in aqueous phase. This hot emulsion (above the melting point of the API/lipid mixture) is then passed through a high-pressure homogenizer such as those manufactured by Microfluidics, using repeated passes to obtain the nanosized emulsion with desired droplet size distribution. This emulsion is then cooled to harden the nanoparticles. The nanoparticles can be lyophilized or spray dried to form a dry powder suitable for loading to a DPI delivery device.
B) SuperCritical CO2 process: Cannabinoids and excipients are soluble in super-critical CO2 as this is the preferred method of extraction. The API and excipients are added to the supercritical CO2 chamber, to which super critical CO2 is added in order to dissolve the API and excipients. When fully dissolved, the mixture is discharged at high pressure through a venture nozzle, producing a dry power of nanoparticles that is collected and loaded into a DPI delivery device.
C) Solvent evaporation process: This process requires API to be soluble in lactide polymers in the solid state (ie no tendency to recrystallize over time in the nanoparticle). The API and lactide polymer are dissolved in a common organic solvent such as methylene chloride. The solution is emulsified in an aqueous solution of pharmaceutically acceptable emulsifiers and emulsified under high shear mixing to form a pre-emulsion. This pre-emulsion is then homogenized in a high pressure homogenizer until the required droplet size distribution is obtained. Afterwards the methylene chloride is removed by evaporation to yield a dispersion of nanoparticles. The lactide polymer is hydrolytically unstsable, and the dispersion must be dried quickly to prevent degradation of the particles. The nanoparticle dispersion can be lyophilized or spray dried to obtain a dry powder of nanoparticles suitable for loading to a DPI delivery device.
D) API nanoparticles: Nanoparticles of API can be formed by high energy milling of coarse API powder in a suitably chosen aqueous phase containing pharmaceutically appropriate colloid stabilizer(s). The final nanodispersion of API crystals can be lyophilized, with added lyoprotectants such as sugars, to form a dry nanopowder of API that can be loaded to DPI devices.

Although inhaler packaging figures significantly in aspects of the present invention, provision is also made herein for alternative packaging, including blister type packaging as illustrated in the drawings appended hereto.

### BRIEF DESCRIPTION OF DRAWINGS

FIG. 1 is a plan view of the top and bottom panels of a high oxygen barrier polymer composite laminate pharmaceutical blister package in which the bottom panel contains molded dose compartments in accordance with an embodiment of this invention. FIG. 1 depicts a side view of a pharmaceutical blister package consisting of an upper, low oxygen permeable flat composite laminate sheet whose exterior surface is comprised of aluminum foil, whose middle layer is comprised of high oxygen barrier polymer and whose inner layer is comprised of oxygen scavenging polymer and a lower composite laminate sheet of the same composition which contains multiple individual molded dose compartments wherein the oxygen scavenger polymer layer forms the inside of each molded dose compartment.

FIG. 1 “A” depicts a side view of a Package 35 consisting of a separate flat upper sheet of composite polymer of aluminum foil 20, oxygen barrier polymer 30 and oxygen scavenger polymer 40 and a separate lower sheet of the same composite polymer containing individual moulded dose compartments 100 wherein oxygen scavenger polymer layer 40 forms the inside of each moulded dose compartment 100. Each dose compartment 100 contains a sachet of oxygen scavenger 41 and a dose of sensitive API 60 in close proximity or in direct contact with the two oxygen scavengers. FIG. 1 “B” depicts a side view of a sealed Package 35, consisting of an upper sheet of composite laminate heat sealed to a lower sheet of similar composite laminate that is formed into low O2 permeable dose pockets 100. Oxygen scavenging polymer 40 must comprise the two inner layers of sealed package 35 and be adjacent to or in direct contact with packaged cannabinoid 40 to scavenge residual head space oxygen and oxygen that over time may permeate through the semi-permeable composite laminate package 35, conferring protection against oxidative degradation of cannabinoids thereby extending their shelf life from 20 days to 60 days.

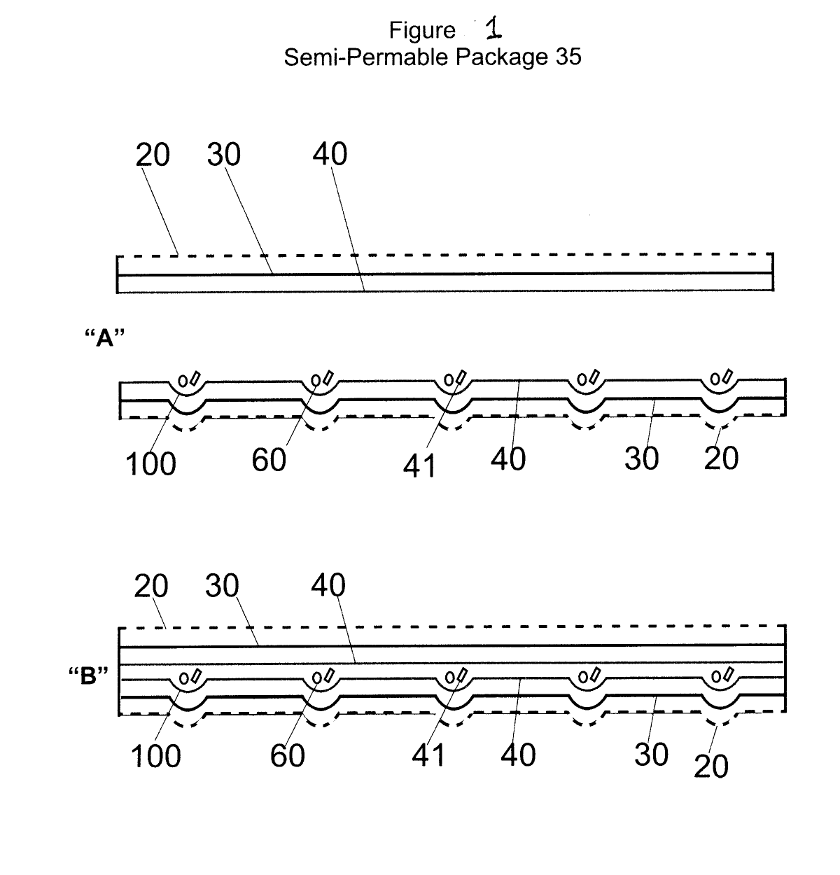

In another embodiment, package 35, may contain an additional oxygen scavenger sachet 41 placed in cavity pocket 100 of laminate package 35 or be adhered to oxygen scavenger polymer 40 so that oxygen scavenger 41 is in close proximity or direct contact with packaged cannabinoid 60 such that head space oxygen or oxygen that over time may permeate semi-permeable package 35 conferring protection from oxidative degradation of cannabinoids and other API and extending the shelf life of package 20 cannabinoids up to 60 days.

In a preferred embodiment of Package 35, prior to sealing the top panel 10 to the formed bottom panel 10A of package 35, cannabinoid is placed in the cavity pocket beside oxygen scavenger 41 in a modified gas atmosphere while the top and bottom panels of package 35 are adhered together in a gas tight manner by heat sealing or adhesive to form the final Package 35. This method extends cannabinoid shelf life by reducing head space oxygen and moisture during the fill-seal-cut process. Residual head space oxygen or oxygen that remains resident in the head space or permeates through semi-permeable package 35 is scavenged by oxygen scavengers 40 and 41. In this manner, package 35 protects sensitive cannabinoids from light, oxygen and water vapour, reducing degradation and extending shelf life of packaged cannabinoids up to 60 days.

A Preferred Formulation: A particularly preferred form of the present invention comprises a formulation (ANA-131), of a CBD, CBG predominant preparation with CBD/THC ratio being equal. Addition of β carophylline, limonene and linalool at concentrations of 0.05% each improved nasal absorption and synergistically increased the efficacy of ANA-131 in treatment of IBS via its targeted action on CB1 and CB2 receptors. Cannabinoids and terpenoids proved to be highly bio-available with an average pulmonary uptake of 70% and intra nasal uptake of over 80%. Both routes avoid first pass hepatic uptake. From earlier studies, it was found that the inclusion of 0.05% limonene and pinene increased the absorption of ANA-131 through the nasal mucosa. The nasal inhaler doses of ANA-131 ranged from 2 mg/dose to 16 mg/dose. In these small trials, the most effective dose was 8 mg/dose BID. It appears that the Cannabis plant is not just a carrier for the active cannabinoids. The combination of “active” and ‘inactive’ synergists are responsible for the “entourage effect” seen when comparing the activity of pure THC from Cannabis extract vs. the same amount of pure THC—which also suggests why greater observed effects of whole extract on vs pure THC—CBD purified combinations in the treatment of IBS. It is believe, without wishing to be necessarily bound, hat one or more of several synergistic interactions arise between cannabinoids and terpenoids: (a) potentiation; (b) antagonism; (c) improved dissolution, solubility, bioavailability; (d) improved anti-bacterial action; (e) modulation of complex adverse events in IBS. In any case, there are biochemical, pharmacological and phenomenological distinctions observed between Cannabis ‘strains’ related to the relative content and ratios of cannabinoids and terpenoids, and this too suggests a botanical basis for the observed synergistic effects seen in IBS treatment with different ratios of phytocannabinoids and terpenoids. A blend of CBD, THCV, CBCR, CBV and terpenoids is an effective anti-inflammatory agent to control joint inflammation with no THC. With the addition of THC=CBD, the blend is 10× more effective than cortisone and 20× more effective than NSAID's without the serious adverse effects (heart attacks and strokes) associated with inhibition of COX-1 or COX-2 enzymes by NSAID's. Caryophyllene is a selective full agonist of CB2, synergistic with the cannabinoid-terpenoid blend in Anandamide hence it is included therein to increase efficacy. Given the lack of psychoactivity of CB2 agonists, caryophyllene offers great promise as a therapeutic compound. This is an example of true synergy as the THC-cannabinoid-terpenoid combination provides a greater effect than the sum of the effects of THC and the other cannabinoids and terpenes separately.

### IBS Treaments:

A cohort of 5 IBS subjects diagnosed with IBS-C, IBS-D or intractable IBS and who had not found a satisfactory treatment modality inhaled 8 mg doses of ANA-131 BID for 180 days. 4 of 5 subjects reported a reduction in IBS symptoms and diminution of depression as scored using the Hamilton Depression scale. The results indicated 4 of 5 subjects Hamilton Depression Scores had returned to normal and the subjects felt they were emotionally able to discontinue their antidepressants.

One subject, a previously healthy 66 year old male who had been diagnosed with advanced prostate cancer. He received 1 year of lutenizing hormone blocking agent (Zoladex) therapy and 42 E-beam radiation treatments (69.7 Grey units of radiation) 80% of which were directed toward the abdomen. During recovery, the subject developed radiation induced colitis with a galaxy of symptoms similar to IBS-A. He was treated with 8 mg ANA-131 BID for 45 days at which time the supply of clinical trial nasal inhalers became exhausted. After a week without treatment, the IBS symptoms returned. The subject then began to smoke marijuana and has been doing so since then. He reports his radiation induced colitis has not returned in the one year period following the end of treatment.

### Alcoholism

A cohort of 6 alcoholic subjects (defined as imbibing 14 oz per day ethanol) inhaled 8 mg doses of ANA-131 twice a day for 30 days. 5 of 6 subjects reported a reduction in alcohol related craving and drinking. This suggests ANA-131 can modulate the reinforcing properties of alcohol and could be a useful adjunct in treatment of chronic alcohol addiction, alcohol withdrawal and alcoholism treatment relapses.

The present invention also extends to a co-therapeutic combination comprising a subclinical dose of morphine together with a compensatory dose of one or more cannabinoid receptor agonist(s) and or antagonist(s).

The combination of cannabinoids and morphine in cannabinoid-morphine SLP for intra nasal inhalation decreases morphine's addiction potential, respiratory depression, opiate-induced constipation and reduces the dose of morphine required. This combination provides synergistically enhanced analgesia for near immediate pain relief in management of severe break-through pain in terminal cancer, post-surgical recovery, cholecystitis, cholelithiasis, pancreatitis, renal calculi, polymyalgia rheumatica, myofascial neurogenic pain and intractable neurogenic pain syndrome. The synergistic effects of the morphine-cannabinoid combination is quite significant and specific as morphine acts directly on its endogenous morphinan receptors, whereas cannabinoids act directly on their endogenous CB1 and CB2 receptors. Such a method offers a significant advance in the pharmaceutical art of cannabinoids and opiates.

In a case of bBreakthrough pain control in terminal cancer, a 68 year old male subject was diagnosed with terminal pancreatic cancer. Palliative treatment consisted of a liquid diet and 20 mg morphine q4 h. Constipation was a minor side effect compared to the loss of reasoning, violence and paranoid ideation the subject was suffering from. The subject was weaned off high dose morphine as 125 mg THC dosed cigarettes replaced the morphine to control break through pain. Cigarettes dosed with 125 mg of THC contained in a Cannabis extract was tried and produced better results than the THC alone. This is due to the entourage effect other cannabinoids in the extract exert on THC, synergistically increasing its analgesic efficacy while modulating its psychotropic side effects. Cigarettes were smoked ad libitum. This regimen proved highly effective in controlling breakthrough pain, improved the subjects communication abilities, arrested paranoid ideation and improved the overall level of comfort of the subject.

In yet another aspect of the present invention, there is provided a co-therapeutic combination comprising a subclinical does or methadone together with a compensatory dose of one or more cannabinoid receptor agonist(s) and of antagonist(s). The combination of cannabinoids and methadone is unique as together they can reduce the significant addiction liability, respiratory depression and constipation associated with methadone, but retain and improve the control of opiate withdrawal and craving and act as a superior substitute for Methadone use in opiate addiction maintenance programs. Methadone blocks the acute symptoms of withdrawal in opiate addicts, but with a cost, as methadone is a dysphoric whose effects are generally considered as unpleasant. In large part, this is the reason for the limited success of methadone maintenance programs. By combining methadone and cannabinoids, the dysphoric effect of methadone is replaced by a mild feeling of well-being. The synergy between cannabinoid and methadone improves the addicted patient's experience with methadone, making the combination a more effective substitute treatment for opiate addiction than methadone alone. Methadone can be readily repurposed with cannabinoids as cannabinoid-methadone SLP for nasal inhaler administration. In a poorly controlled study undertaken in a street drug clinic setting, the combination reduced acute withdrawal symptoms with one-half the dose of methadone and was more effective than methadone alone in weaning opiate addicts from their opiate dependence. Such an invention is suitable for use in chronic maintenance treatment programs and would be a desirable replacement for the significantly larger methadone dose currently employed at street clinics. Such a method offers a significant advance in the pharmaceutical art of the cannabinoids.

In opiate addiction therapy trials, a cohort of 6 opiate addicted subjects on Methadone maintenance programs inhaled 8 mg dose of ANA-131 twice a day for 30 days. All subjects reported a reduction in opiate craving, opiate-related stimuli and opiate use. This suggests ANA-131 can effectively modulate the reinforcing properties of opiates and could be a useful adjunct in the treatment of opiate addiction. An unexpected side effect of the ANA-131 treatment was the subjects reported that although they still took Methadone periodically, the effects of ANA-131 were preferred over those of Methadone.

1. An inhaler-delivery-device-packaged homogenate of solid heterogeneous-lipid particulates carrying lipophilic cannabinoid receptor agonists and/or antagonists, said solid heterogeneous-lipid particles comprising:
a. one (or more) lipid(s) whose melting point(s) is (are) substantially above room temperature; in combination with,
b. one (or more) lipid(s) whose melting point(s) is (are) substantially less than room temperature.

2. The inhaler-delivery-device-packaged homogenate according to claim 1, comprising solid lipid particle of a homogenate selected from the group comprising:
a. Solid lipid particle homogenate based on a compounded excipient comprised of a formulation of mutually compatible lipids including a first lipid having a melting point substantially greater than room temperature, and a second lipid having a melting point substantially below room temperature; or,
b. Solid lipid particle homogenate of lipid phytoextracts fats/oils containing a first lipid having a melting point substantially greater than room temperature, and a second lipid having a melting point substantially below room temperature; or,
c. A combination thereof.

3. The inhaler-delivery-device-packaged homogenate according to claim 2 wherein said first lipid comprises one or more saturated fatty acid(s), and said second lipid comprises one or more unsaturated fatty acid(s).

4. The inhaler-delivery-device-packaged homogenate according to claim 3, wherein said first lipid includes one or more of the group of unsaturated fatty acids comprising palmitic acid and stearic acid.

5. The inhaler-delivery-device-packaged homogenate according to claim 3, wherein said second lipid includes one or more of the group of saturated fatty acids comprising oleic acid and linoleic acid.

6. The inhaler-delivery-device-packaged homogenate according to claim 2, is a solid lipid particle homogenate of lipid phytoextracts fats/oils.

7. The inhaler-delivery-device-packaged homogenate according to claim 6, wherein said solid lipid particle homogenate of lipid phytoextracts fats/oils comprises one or more of the group selected from solid lipid particle homogenate of extracted cannabis fats/oils; or, solid lipid particle homogenate of one or more vegetable oils.

8. The inhaler-delivery-device-packaged homogenate includes a solid lipid particle homogenate of extracted cannabis-endogenous fats/oils, and further comprises cannabis-endogenous essential oils.

9. The inhaler-delivery-device-packaged homogenate according to claim 8, wherein said essential oils are cannabis phytoterpenoids, and include one or more of the group selected from limonene, myrcene, α-pinene, linalool, β-caryophyllene, caryophyllene oxide, nerolidol and phytol.

10. The inhaler-delivery-device-packaged homogenate according to claim 1, wherein said device is selected from one of the group comprising: propellant or inspiration of dry solid lipid particulate homogenate devices; or “wet” pumped aerosols of solid lipid particle homogenate devices.

11. The inhaler-delivery-device-packaged homogenate according to claim 10, wherein said device is a “wet” pumped aerosols of solid lipid particle homogenate device and said solid lipid particles include one or more of the group selected from microparticles and nanoparticles.

12. A lipophilic active pharmaceutical ingredient, lipophilic bioactive nonessential nutrient agent or lipophilic essential nutrient, in combination with a carrier comprised of mutually compatible lipids including a first crystalized lipid providing a crystalline structure with a second lipid interstitially disposed within said structure and in close-packing-interfering relation with first lipid crystals thereof, and wherein said first lipid is a solid at room temperature, and said second lipid is a liquid at a temperature of about 21 degrees centigrade.

13. The combination according to claim 12, wherein said first lipid has a melting point higher than normal internal human body temperature
i. *Note for description: or higher: must be low enough so that its melting does not result in substantial damage to a temperature labile API, e.g. cannabinoid.

14. The combination according to claim 12, wherein said first lipid is stearic acid and said second lipid is sunflower oil.

15. The combination according to claim 12, wherein the ratio of said first lipid to said second lipid is about 70 parts to 30 parts by weight.

16. The combination according to claim 12, further comprising an antioxidant.

17. The combination according to claim 16, wherein the antioxidant is alpha tocopherol

18. The combination to claim 12, in an aqueous excipient-in-water emulsion including a surfactant.

19. The combination according to claim 18, wherein said emulsion is a polysorbate surfactant emulsion.

20. The combination according to claim 18 comprising a pre-homogenate aqueous, emulsifier-stabilized, uniform emulsion of liquid phase first and second excipient lipids.

21. The combination according to claim 18, comprising a homogenate of said pre-homogenate aqueous emulsion.

22. The combination according to claim 21, at a temperature below the melting point of at least said first lipid, and formed of solid lipid particulates of excipient-borne active pharmaceutical ingredient.

23. The combination according to claim 22, wherein said solid lipid particulate excipient-borne active pharmaceutical ingredient includes a substantial proportion of numbers of particles in the micrometer and/or nanometer size ranges.

24. The combination according to claim 23, wherein said particulates form a dry, friable powder.

25. The combination according to claim 24, wherein said particulates are lyophilized.

26. The combination according to claim 12, wherein said lipophilic active pharmaceutical ingredient, lipophilic bioactive nonessential nutrient agent or lipophilic essential nutrient, comprises 30 to 96% by weight of said combination.

27. A packaged product comprising the combination according to claim 12.

28. A packaged product comprising the combination according to claim 27, wherein said package is one of the group selected from dry dispense packaging; wet pump dispense packaging, blister packaging; gel cap dispensing.

29. The packaged product according to claim 28, wherein said package is a metered dose dispenser.

30. The packaged product according to claim 28 is an intra nasal dispenser.

31. The combination of claim 12, wherein said lipophilic active pharmaceutical ingredient, lipophilic bioactive nonessential nutrient agent or lipophilic essential nutrient is one or more of the group selected from the lipophilic vitamins; opiates, endogenous cannabinoids, synthetic cannabinoids, solvent extracted (eg especially ethanol extracted) phytocannabinoids from (e.g. and preferably cannabis via carbon dioxide extracted) plant extracts, essential oils of cannabis plant cannabinoids and/or terpenoids, a cannabinoid receptor agonist, and cannabinoid receptor antagonist.

32. The combination of claim 31, wherein the API includes an opiate.

33. The combination of claim 32, wherein the opiate is selected from the group comprising methadone or morphine.

34. The combination of claim 32 or 33, comprising an opiate with a (phyto-cannabinoid extract predominantly containing) a THC/cannabidiol combination (with minor proportions of other phyto-canabinoids and/or phyto-terpenoids (or synthetic equivalents thereof)) (“entourage effect” is the sum of/between multiple synergies), wherein the proportion of opiate is a moderated dose in proportion to a moderating effect of the phyto-cannabinoid extract. (subclinical opiate doses)

35. The combination according to claim 34, wherein said API comprises 30 to 96% by weight of said API and lipid excipient combination.

36. A method for producing a solid lipid particle pre-homogenate, comprising:
a. heating a mixture comprising:
i. a heterogeneous lipid combination including:
1. one (or more) lipid(s) whose melting point(s) is (are) substantially above room temperature; in combination with,
2. one (or more) lipid(s) whose melting point(s) is (are) substantially less than room temperature, and
ii. one or more of a group selected from lipophilic API, lipophilic bioactive nonessential nutrient, or lipophilic essential nutrient
b. to above the melting point which is substantially above room temperature sufficient to melt said lipids and reduce said mixtures viscosity;
c. pre-homogenizing said heated mixture to produce a stable pre-homogenate.

37. The method according to claim 35, further comprising the addition of surfactant stabilizer to said mixture.

38. The method according to claim 36, wherein said surfactant is a non-ionic surfactant, preferably selected from the group consisting of polysorbates or poloaxmers.

39. The method according to claim 35 wherein said mixing is carried out for about 10 minutes at about 20,000 rpm.

40. A method of preparing a solid lipid particle homogenate comprising heating/homogenizing the heated pre-homogenate according to claim 35, at about 500 to 1500 bar at least once and preferably twice to produce a further heated homogenate, and then cooling the heated homogenate to about room temperature, to produce a solid lipid homogenate.

41. The method according to claim 39, wherein said homogenization carried out to produce solid lipid microparticles and/or nanoparticles in said room temperature homogenate.

42. The method according to claim 40, wherein said room temperature homogenate is then lyophilized.

43. The method according to claim 35 wherein said mixture comprises a cannabis carbon dioxide extract wherein said heterogeneous lipids are comprised of cannabis fats and oils from said cannabis extract.

44. The method according to claim 35, wherein a selected API comprises a carbon dioxide cannabis extract, containing cannabis extracted phytocannabinoids.

45. The method according to claim 43 wherein said cannabis extracted phytocannabinoids is a carbon dioxide cannabis extract residual following ethanolic extraction thereof, and said heterogeneous lipid combination is comprised of lipids from sources other than cannabis.

46. A co-therapeutic combination comprising a subclinical dose of morphine together with a compensatory dose of one or more cannabinoid receptor agonist(s) and or antagonist(s).

47. A co-therapeutic combination comprising a subclinical does or methadone together with a compensatory dose of one or more cannabinoid receptor agonist(s) and of antagonist(s).
